(19) United States
(12) Patent Application Publication
(10) Pub. No.: US 2025/0107750 A1
(43) Pub. Date: Apr. 3, 2025

(54) POSTURE EVALUATION APPARATUS, POSTURE EVALUATION SYSTEM, POSTURE EVALUATION METHOD, AND COMPUTER READABLE MEDIUM

(71) Applicants: NEC CORPORATION, Tokyo (JP); National University Corporation Tokyo Medical and Dental University, Tokyo (JP)

(72) Inventors: Shuhei NOYORI, Tokyo (JP); Makoto YASUKAWA, Tokyo (JP); Keisuke SUZUKI, Tokyo (JP); Yuki KOSAKA, Tokyo (JP); Akimoto NIMURA, Tokyo (JP); Koji FUJITA, Tokyo (JP); Takuya IBARA, Tokyo (JP)

(73) Assignees: NEC CORPORATION, Tokyo (JP); National University Corporation Tokyo Medical and Dental University, Tokyo (JP)

(21) Appl. No.: 18/892,763

(22) Filed: Sep. 23, 2024

(30) Foreign Application Priority Data
Sep. 29, 2023 (JP) 2023-170016

(51) Int. Cl. A61B 5/00; A61B 5/11

(52) U.S. Cl. CPC A61B 5/4561; A61B 5/1114; A61B 5/742

(57) ABSTRACT

To provide a posture evaluation apparatus, a posture evaluation system, a posture evaluation method, and a program that are capable of suitably evaluating a backward-bending posture. The posture evaluation apparatus according to the present disclosure calculates a feature including at least a feature of an inclination of a head relative to a trunk, based on body information including information indicating at least an inclination of the trunk and an inclination of the head in a body of a subject person in a backward-bending posture in which the subject person is bent backward, and estimates a state of at least a spine in the backward-bending posture, based on the calculated feature.

### INCORPORATION BY REFERENCE

This application is based upon and claims the benefit of priority from Japanese patent application No. 2023-170016, filed on Sep. 29, 2023, the disclosure of which is incorporated herein in its entirety by reference.

### TECHNICAL FIELD

The present disclosure relates to a posture evaluation apparatus, a posture evaluation system, a posture evaluation method, and a program.

### BACKGROUND ART

In recent years, with spread of online training and self-training, there is an increasing need for ordinary people without specialized knowledge to evaluate own postures.

Japanese Unexamined Patent Application Publication No. 2000-207568 describes a posture measurement apparatus intended for easily measuring a posture of an object to be measured. The measurement apparatus described in Japanese Unexamined Patent Application Publication No. 2000-207568 estimates a position, a size, and a posture of an object to be measured in a posture measurement image being an image of the object to be measured captured by a camera. The measurement apparatus described above includes a posture-measuring means for determining, from a posture measurement image and an estimation result, a centerline of an outline of the object to be measured in the posture measurement image, and measuring the posture of the object to be measured, based on an inclination of the centerline. The posture-measuring means described above includes an edge-extracting means, a centerline-estimating means, and a centerline-determining means. The edge-extracting means described above extracts edges of the posture measurement image to generate an edge image. The centerline-estimating means described above estimates a centerline of the outline of the object to be measured in the posture measurement image from an estimation result. The centerline-determining means described above takes an estimated centerline from the centerline-estimating means and an edge image from the edge-extracting means as inputs and determines the centerline from edges near the estimated centerline in the edge image.

However, regarding a backward-bending posture of a subject person especially when wearing clothes, the measurement apparatus described in Japanese Unexamined Patent Application Publication No. 2000-207568 is not capable of measuring and evaluating the backward-bending posture of an object to be measured relatively easily and with high accuracy, because the clothes hide an actual shape of the back.

### SUMMARY

An example object of the present disclosure is to provide a posture evaluation apparatus, a posture evaluation system, a posture evaluation method, and a program that are capable of suitably evaluating a backward-bending posture.

In a first example aspect of the present disclosure, a posture evaluation apparatus includes: a feature calculating unit configured to calculate a feature including at least a feature of an inclination of a head relative to a trunk, based on body information including information indicating at least an inclination of the trunk and an inclination of the head in a body of a subject person in a backward-bending posture in which the subject person is bent backward; and a state-estimating unit configured to estimate a state of at least a spine in the backward-bending posture, based on the feature.

In a second example aspect of the present disclosure, a posture evaluation system includes a posture evaluation apparatus and a terminal apparatus capable of communicating with the posture evaluation apparatus, and the posture evaluation apparatus includes: a feature calculating unit configured to calculate a feature including at least a feature of an inclination of a head relative to a trunk, based on body information including information indicating at least an inclination of the trunk and an inclination of the head in a body of a subject person in a backward-bending posture in which the subject person is bent backward, the body information being acquired by the terminal apparatus; and a state-estimating unit configured to estimate a state of at least a spine in the backward-bending posture, based on the feature.

In a third example aspect of the present disclosure, a posture evaluation method includes, by a posture evaluation apparatus: calculating a feature including at least a feature of an inclination of a head relative to a trunk, based on body information including information indicating at least an inclination of the trunk and an inclination of the head in a body of a subject person in a backward-bending posture in which the subject person is bent backward; and estimating a state of at least a spine in the backward-bending posture, based on the feature.

In a fourth example aspect of the present disclosure, a program causes a computer to execute posture evaluation processing of: calculating a feature including at least a feature of an inclination of a head relative to a trunk, based on body information including information indicating at least an inclination of the trunk and an inclination of the head in a body of a subject person in a backward-bending posture in which the subject person is bent backward; and estimating a state of at least a spine in the backward-bending posture, based on the feature.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 is a block diagram illustrating a configuration example of a posture evaluation apparatus of the present disclosure;

FIG. 2 is a block diagram illustrating another configuration example of the posture evaluation apparatus of the present disclosure;

FIG. 3 is a drawing illustrating an example of an image captured by an image-capturing unit of the posture evaluation apparatus according to the present disclosure;

FIG. 4 is a drawing illustrating an example of a key point according to the present disclosure;

FIG. 5 is a drawing illustrating an example of a line segment connecting key points according to the present disclosure;

FIG. 6 is a drawing illustrating an example of the feature according to the present disclosure;

FIG. 7 is a drawing illustrating another example of the feature according to the present disclosure;

FIG. 8 is a drawing illustrating an example of a state label output as estimation results of the state according to the present disclosure;

FIG. 9 is a drawing illustrating an example of an image displayed on a display unit according to the present disclosure;

FIG. 10 is a drawing illustrating another example of the image displayed on a display unit according to the present disclosure;

FIG. 11 illustrates another example of an image displayed on the display unit of the present disclosure;

FIG. 12 is a drawing illustrating another example of the image displayed on a display unit according to the present disclosure;

FIG. 13 is a flowchart describing a posture evaluation method according to the present disclosure; and

FIG. 14 is a drawing illustrating a configuration example of a posture evaluation system according to the present disclosure.

### EXAMPLE EMBODIMENT

### First Example Embodiment

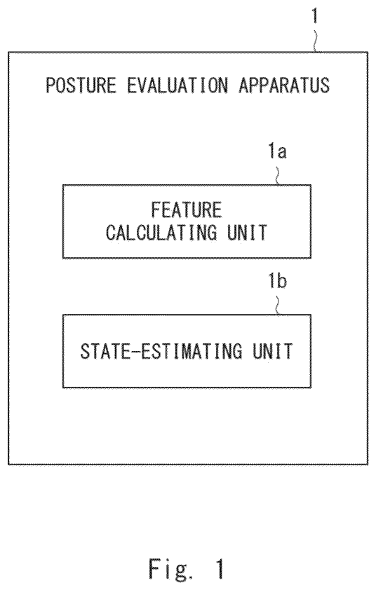

Referring now to FIG. 1, a configuration example of a posture evaluation apparatus 1 will be described below.

The posture evaluation apparatus 1 includes a feature calculating unit 1a and a state-estimating unit 1b, as illustrated in FIG. 1.

The feature calculating unit 1a calculates a feature including at least a feature of the inclination of the head relative to the trunk based on body information including information indicating at least the inclination of the trunk and the inclination of the head in the body of the subject person in the backward-bending posture in which the subject person is bent backward. Information indicating the inclination of a part, such as the trunk, the head, and the like of the body can be, for example, the angle or the positions of two ends of such a part.

As described herein, the term “inclination” refers to the inclination of the subject person when viewed from the side, that is, the inclination in a sagittal plane, or in other words, the inclination in the direction along the sagittal plane. The following description is based on such an assumption, but the process may also include the inclination in the coronal plane in the backward-bending posture. The description about the positions described below will be given assuming that it refers to the position in the sagittal plane, but the process may include the positions in the coronal plane in the backward-bending posture.

The body information can also include a captured image of the side surface of the body of the subject person in the backward-bending posture. Capturing the image of the side surface of the body of the subject person refers to capturing the image of the subject person from a direction perpendicular to the sagittal plane of the subject person. This image can be obtained, for example, by capturing images of the side surface of the body of the subject person with a camera in a smartphone or the like. In this manner, the feature calculating unit 1a can calculate features using such images. The images to be included in the body information can include images of the body of the subject person in the backward-bending posture captured from the opposite side or from directions other than the side.

Alternatively, the body information can include information extracted from the images described above. In other words, the feature calculating unit 1a can calculate the feature using the information extracted from such images. The information to be extracted includes, for example, one or both of position information indicating the position of body keypoints and the position of key points other than the body keypoints of the body. As a matter of course, the body information can include both the images and information extracted from the images. Examples of the images described above, and information extracted from the images described above will be explained in second example embodiment. Examples of the information extracted from the images described above include, for example, position information indicating the position of a predetermined part of the body that can be estimated from the body keypoints.

The feature calculated by the feature calculating unit 1a should be at least a feature that indicates the feature of the inclination of the head relative to the trunk. For example, the calculated feature can be expressed in terms of values such as the angle of the straight line in the direction of inclination of the head relative to the straight line in the direction of inclination of the trunk itself, or the level value of that angle when assigned to one of several levels by a threshold process or the like.

The feature calculating unit 1a may also use a learning model obtained by machine learning from data of many subject persons to input the body information and output the feature. Algorithms or the like of this learning model is not limited, and any types of algorithms can be employed for this learning model, including linear regression models and support vector machines.

The state-estimating unit 1b estimates the state of at least the spine in the backward-bending posture, that is, the state of at least the spine in the backward-bending posture, based on the feature calculated by the feature calculating unit 1a. The target of the estimation can include the state of one or more of various parts, for example, the upper thoracic spine, the lower thoracic spine, the lumbar spine, the lumbosacral transition region, the hip joints, the upper extremity, the scapular spine, the anterior superior iliac spine, the knee joints, and the ankle joints. The estimation of the state of these parts will be illustrated in second example embodiment and onwards.

For example, the state-estimating unit 1b stores in its internal memory a correspondence table that maps features to the states of the spine and the like in the backward-bending posture in advance, so that, by referring to the correspondence table, the states of the spine and other parts in the backward-bending posture corresponding to the feature received from the feature calculating unit 1a may be searched for.

The state-estimating unit 1b may also estimate the state by using a learning model obtained by machine learning from data of many subject persons, inputting features, and outputting the states of the spine and the like in the backward-bending posture. Algorithms of this learning model is not limited, and any types of algorithms can be employed for this learning model, including linear regression models and support vector machines.

In this manner, posture evaluation apparatus 1 is an apparatus that evaluates backward-bending posture based on the body information. Even when the subject person is clothed and the body information does not include information on the inclination or position of the spine, the posture evaluation apparatus 1 can estimate the state of the spine in a backward-bending posture for the following reason. That is, even in such a case, the posture evaluation apparatus 1 can estimate the state of the spine in the backward-bending posture based on alternative movements in parts other than the spine, such as the inclination of the trunk and head caused by the mobility of the spine.

In fact, the spine shape can be estimated using the shape of the back, which is easily observed from the body surface. However, if the subject person is wearing clothes, the clothes hide the shape of the back in the backward-bending posture. The sagging of the clothes hides the shape of the back considerably, especially if the clothes are not the type that closely fits the subject person. Therefore, in such cases, accurately evaluating the backward-bending posture for evaluating the range of spine extension, that is, an extension posture without employing first example embodiment would be difficult with a process based solely on the shape of the back. therefore, to achieve such an accurate evaluation without employing first example embodiment, expensive specialized equipment is required as in methods such as a method using a depth camera, a method using an acceleration sensor, or a method that scans along the spine with a measuring element. In other words, without such expensive specialized equipment, the accuracy of posture evaluation will not be as good as the accuracy of posture evaluation by specialists such as therapists, trainers, and the like. However, specialists such as therapists, trainers, and the like evaluate the balance of the spine as a whole in terms of bending, not the position or inclination of individual vertebrae. Therefore, for the purpose of enabling the general public to achieve professional-level evaluation of one's posture, there is no need to aim for the same level of accuracy as the above-described methods that require specialized equipment described above.

On the other hand, the posture evaluation apparatus 1 estimates the state of the spine or the like in a backward-bending posture using information about the head and the trunk or other parts, which are easily recognizable from the appearance and move in tandem with the spine, rather than information about the spine, which is difficult to estimate from the appearance. In other words, the posture evaluation apparatus 1 estimates the state of the spine based not on the shape of the spine itself, but on compensatory movements in parts other than the spine that occur in response to the mobility of the spine.

In this manner, the posture evaluation apparatus 1 can estimate and evaluate the state of the spine or the like in a backward-bending posture at a professional-level, even from information that can be obtained simply as the body information. In other words, the posture evaluation apparatus 1 can estimate and evaluate the state of the spine or the like in a backward-bending posture relatively easily and with high accuracy. In addition, with the spread of online training and self-training, there is a growing need for the general public to evaluate posture and alignment for themselves, and first example embodiment can meet such a need as well.

In particular, by including the image or information extracted from the image described above in the body information, the state of at least the spine in the backward-bending posture can be estimated from information obtained by capturing an image of the side surface of the body with a camera in a smartphone or the like, for example. This allows us to evaluate posture and alignment of the spine in a backward-bending posture, especially part-specifically, by using images obtained with a camera in a smartphone, for example, without the need for specialists such as trainers, therapists, and the like. Therefore, it is relatively easy and accurate to evaluate the state of the spine or the like in a backward-bending posture in situations such as online training and self-training. First example embodiment can also be used for self-training or online training by the subject person himself/herself, with or without the advice of trainers, therapists, and the like. First example embodiment can also be used for simple screening by trainers, therapists, and the like prior to treatment of the subject person.

As mentioned above, the posture evaluation apparatus 1 is capable of evaluating, among other postures, the backward-bending posture. However, the posture evaluation apparatus 1 may be equipped with a function to evaluate other postures, such as forward-bending posture and standing posture.

### Second Example Embodiment

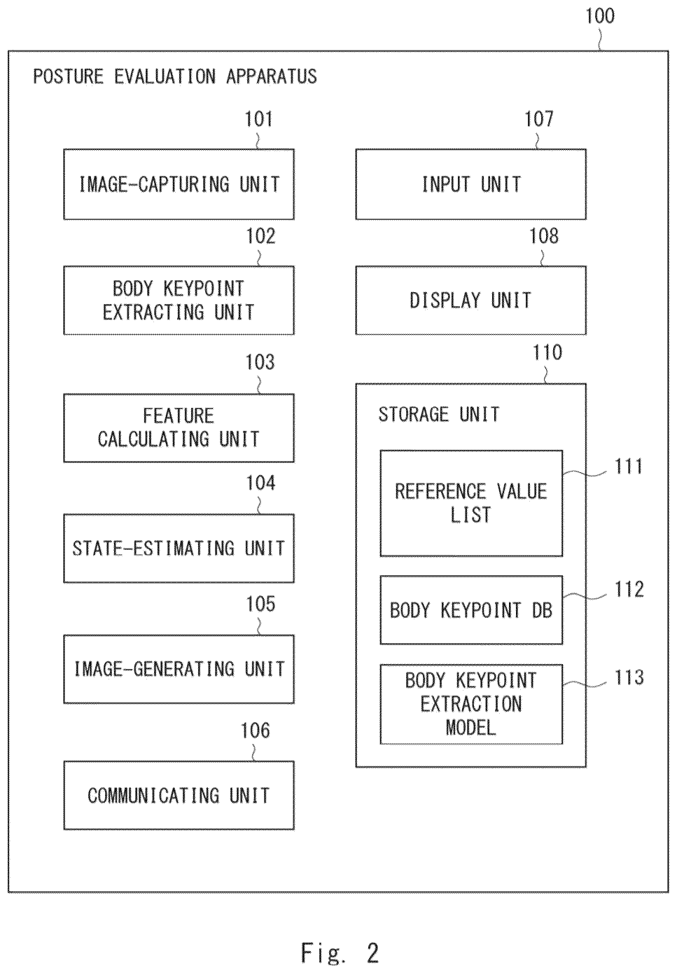

Referring now to FIG. 2, a configuration example of a posture evaluation apparatus 100 will be described.

The posture evaluation apparatus 100 illustrated in FIG. 2 is a user terminal such as a smartphone, a tablet terminal, or a personal computer owned by the user. The user includes both the subject person who is subject to evaluation by the posture evaluation apparatus 100 and the evaluator who evaluates the posture of others using the posture evaluation apparatus 100. When the subject person evaluates his/her own posture using the posture evaluation apparatus 100 in self-training or the like, the subject person serves also as the evaluator. When an evaluator uses the posture evaluation apparatus 100 to evaluate the posture of others, the evaluator would be, for example, a therapist or a trainer.

The posture evaluation apparatus 100 includes an image-capturing unit 101, a body keypoint extracting unit 102, a feature calculating unit 103, a state-estimating unit 104, an image-generating unit 105, a communicating unit 106, an input unit 107, a display unit 108, and a storage unit 110 as illustrated in FIG. 2. The input unit 107 and the display unit 108 may be configured as a single display with touch panel, or each may be provided separately. The storage unit 110 also stores a reference value list 111, a body keypoint database (Database: DB) 112, and a body keypoint extraction model 113. Here, the feature calculating unit 103 and the state-estimating unit 104 are examples of feature calculating unit 1a and state-estimating unit 1b, respectively.

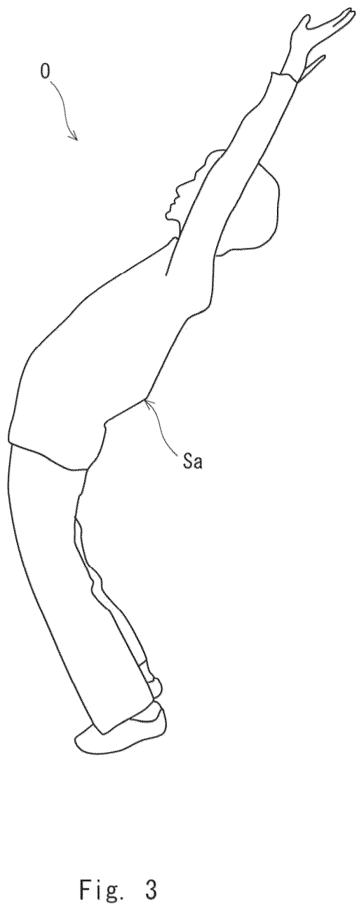

The image-capturing unit 101 acquires images by capturing the image of the side surface of the body of the subject person in a backward-bending posture. An example of the image of the side surface of the body of the subject person captured by the image-capturing unit 101 is illustrated in FIG. 3. The image illustrated in FIG. 3 shows the side of a person O corresponding to the subject person who is bending backward. Here, the image captured by the image-capturing unit 101 is a two-dimensional image and may be a two-dimensional RGB image. The image-capturing unit 101 inputs the captured images to the body keypoint extracting unit 102.

The image-capturing unit 101 may also acquire images by taking movie of the side surface of the body of the subject person in the backward-bending posture. In this case, the user may specify a time point at which the backward-bending posture is to be evaluated by operating the input unit 107. The image captured at the time point specified by the user may then be input to the body keypoint extracting unit 102.

The body keypoint extracting unit 102 extracts position information indicating the position of, for example, the head, the cervical vertebrae, and the hip joints from the images captured by the image-capturing unit 101. In the present disclosure, such position(s) of the target of extraction are also referred to as “key point(s)” and the position information indicating such position(s) is also referred to as “position information of the key points”.

The position information on the image is, for example, image coordinates. As described herein the term “image coordinates” refer to coordinates used to indicate the position of a pixel in a two-dimensional image, in which the center of the leftmost and uppermost pixel in a two-dimensional image is the origin, left and right directions or horizontal direction is the x-direction, and the vertical direction or up and down directions is the y-direction, for example.

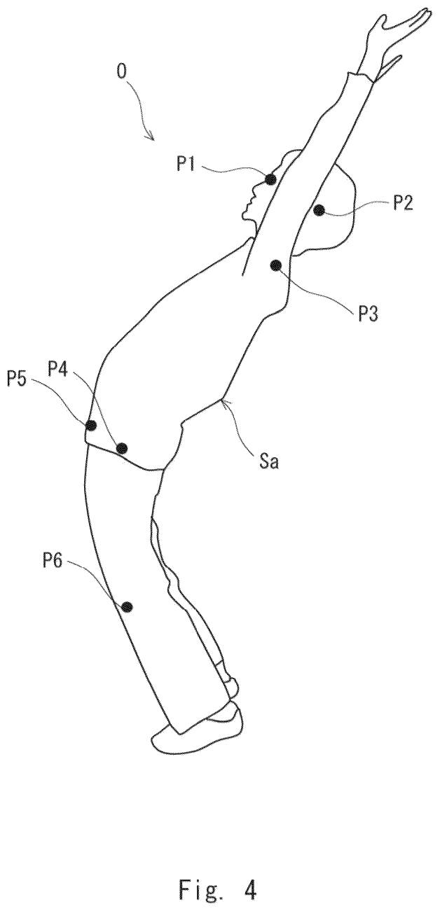

Examples of key points extracted by the body keypoint extracting unit 102 are shown in FIGS. 3 through 5. FIG. 4 illustrates an example of key points P1-P6 extracted by the body keypoint extracting unit 102 from the image illustrated in FIG. 3. In FIGS. 3 to 5, the sign Sa designates a sagged portion of the user's garment.

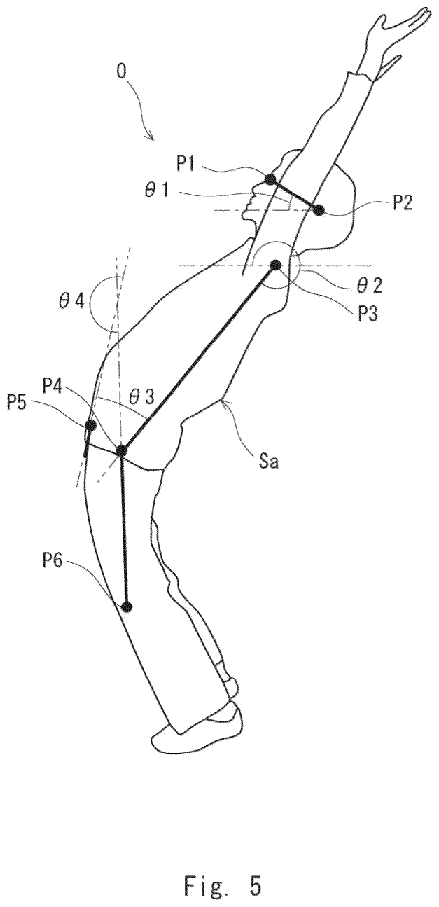

In FIG. 4 and FIG. 5, the sign P1 designates the key point of the eye, the sign P2 designates the key point of the ear, the sign P3 designates the key point of the prominent vertebra, and the sign P4 designates the key point of the hip joints. In FIGS. 4 and 5, the sign P5 designates the key point of the anterior superior iliac spine and the sign P6 designates the key point of the knee joints.

Hereafter, key points of eyes are referred to as “eye key point”. Likewise, similar abbreviations will be used such that key points of ears will be referred to as “ear key point”. Here, the ear key point and the eye key point are used as position information indicating two positions on the head. For example, however, the key point at the center of the head can also be used. The key point of the prominent vertebra is used as position information to indicate the position of the cervical vertebrae.

In this manner, the position information of the cervical vertebrae on the image is described here as being the position information of the prominent vertebra (C7), but it can be the position information of any of the seven vertebrae that make up the cervical vertebrae. The position information of the prominent vertebra is, for example, the position information of the pixel located at the center of the image area corresponding to the prominent vertebra on the image. For other parts, the position information of the pixel located at the center of the part can be used in the same way. For example, if a part is too large to be represented by a single position, position information indicating the position of a pixel at one end or pixels at both ends of the part may be used as the position information for that part.

The body keypoint extracting unit 102 extracts position information of, for example, key points P1-P4 from images captured by the image-capturing unit 101 using the learned body keypoint extraction model 113. In practice, the position of each key point is estimated by the body keypoint extraction model 113, and position information indicating its position is extracted. The posture evaluation apparatus 100 performs machine learning in advance using the body keypoint extraction model 113, which is a machine learning model, and the body keypoint database 112, which is teacher data, to generate a learned body keypoint extraction model 113. Any types of algorithms can be employed for this body keypoint extraction model 113, including, for example, a model using deep learning. Even if the eyes and ears are hidden by the arms in the image, the position information of the eye key point and ear key point can be extracted from the shape of the head.

The position information of the key points may be information expressed in three-dimensional coordinates defined in the x-direction, which corresponds to the left and right directions or horizontal direction, the y-direction, which corresponds to the up and down directions or vertical direction, and the z-direction, which corresponds to the depth direction, of the two-dimensional image captured by the image-capturing unit 101. This can be achieved by using a body keypoint extraction model 113 that extracts position information of the key points represented by three-dimensional coordinates from a two-dimensional image.

The body parts from which the body keypoint extracting unit 102 extracts key points are not limited to the head, the cervical vertebrae, and the hip joints as described above, but may also extract a key point of the shoulder joints, for example. The position of the target of extraction should be the positions of at least several parts of the body that can provide information on the inclination of the trunk and the inclination of the head.

Furthermore, the body keypoint extracting unit 102 calculates information indicating at least the inclination of the trunk and the inclination of the head from the position information of the key points extracted in this manner, for example, by geometric calculation. FIG. 5 illustrates an example of a line segment connecting key points.

For example, the body keypoint extracting unit 102 obtains differences between the coordinates of the eye key point P1 and the ear key point P2 in the x-direction and the y-direction, and obtains those difference values as information indicating the inclination of the head, or performs a geometric calculation from those difference values to obtain an angle θ1 indicating the inclination of the head. Similarly, the body keypoint extracting unit 102 obtains differences between the coordinates of the prominent vertebra key point P3 and the hip joint key point P4 in the x-direction and y-direction and obtains those difference values as information indicating the inclination of the trunk, or performs a geometric calculation from those difference values to obtain the angle θ2 indicating the inclination of the trunk.

Here, both angles θ1 and θ2 are obtained as angles to the horizontal line, but they may also be, for example, angles to a vertical line. Supplementary explanations are provided on how to find vertical line in the image and horizontal line in the image that can be used to calculate such angles and the features described below. Assuming that the image-capturing unit 101 is fixed horizontally in the left and right directions and vertically in the front and back directions, the straight lines parallel to the left and right edges of the image and the top and bottom edges of the image are considered as the vertical line and the horizontal line, respectively. At the same time as capturing a still or moving picture with the image-capturing unit 101, inclination information of the image-capturing unit 101 is acquired using an acceleration sensor or the like installed separately, and vertical line and horizontal line in the key point coordinate system are obtained using such information.

Key points may also be specified by the user operating the input unit 107 while images captured by the image-capturing unit 101 are displayed on the display unit 108. The position information of the key points specified by the user should then be input to the body keypoint extracting unit 102, and the body keypoint extracting unit 102 may calculate information indicating at least the inclination of the trunk and the inclination of the head.

The body keypoint extracting unit 102 inputs the calculated body information, including at least the information indicating the inclination of the trunk and the inclination of the head, to the feature calculating unit 103. In this manner, in second example embodiment, the body keypoint extracting unit 102 extracts position information from the image and passes the extracted position information to the feature calculating unit 103 as at least part of the body information.

The feature calculating unit 103 calculates feature including at least the feature of the inclination of the head in relation to the trunk based on the body information input from the body keypoint extracting unit 102. In this manner, the feature calculating unit 103 does not need to calculate the feature for the spine to be evaluated with the posture evaluation apparatus 100.

The feature calculating unit 103 calculates the feature as the angle formed by any two of the following: a line segment connecting two key points, a straight line indicating the inclination of the specified part, a vertical line in the image, and a horizontal line in the image.

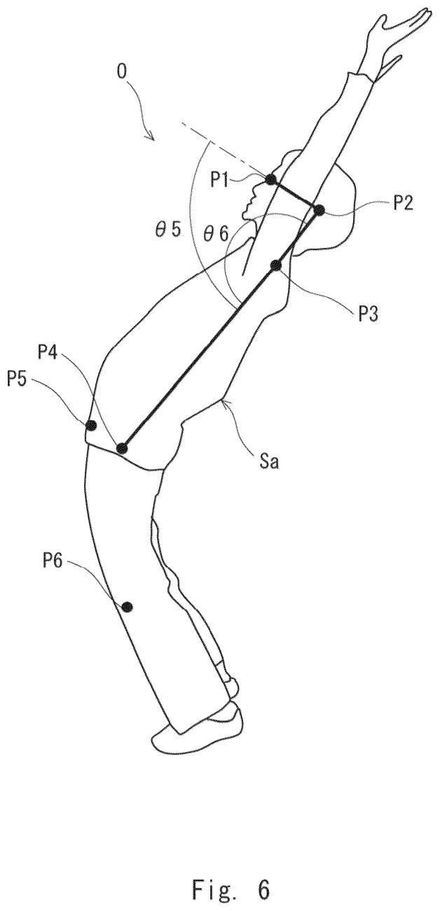

Referring now to FIG. 6, an example of feature calculated by the feature calculating unit 103 will be described. The feature calculating unit 103 calculates the feature, for example, as the angle 05 of the angle between the line segment connecting key points P1 and P2 and the line segment connecting key points P3 and P4. The angle 05 is a feature that indicates the feature of a straight line indicating the inclination of the head relative to a straight line indicating the inclination of the trunk.

Alternatively, the feature calculating unit 103 calculates the feature, for example, as the angle θ6 formed between the line segment connecting key points P2 and P3 and the line segment connecting key points P3 and P4. Angle θ6 is also a feature that indicates the feature of a straight line indicating the inclination of the head relative to a straight line indicating the inclination of the trunk.

In this manner, the body information used to calculate the feature can include information indicating the positions of at least the cervical vertebrae, the hip joints, the eyes, and the ears of the user in the backward-bending posture (referred to as “first position information” for convenience). The feature calculating unit 103 can then calculate the inclination of the trunk and the inclination of the head based on the first position information, and use the calculated inclination of the trunk and the inclination of the head to calculate the feature including at least the feature of the inclination of the head relative to the trunk.

The body information can also include information indicating the position of the head relative to the trunk, that is, the relative position of the head to the trunk, on the user's body in the backward-bending posture. In this case, the feature calculating unit 103 calculates not only the feature of the inclination of the head relative to the trunk, but also the feature of the position of the head relative to the trunk as a feature.

In this example as well, the body information can include the first position information described above. For example, the position of the trunk and the position of the head can be calculated, in the same manner as their inclinations, using the eye key point P1 and the ear key point P2, the prominent vertebrae key point P3 and hip joint key point P4, respectively. Based on the first position information, the feature calculating unit 103 can calculate the inclination of the trunk, the inclination of the head, and the position of the head relative to the trunk to calculate the following feature. In other words, the feature calculating unit 103 can use the calculated inclination of the trunk, inclination of the head, and position of the head relative to the trunk to calculate feature including at least the features of the inclination and the position of the head relative to the trunk.

Even if the subject person is wearing clothes, the shape of the anterior surface of the body can be recognized to some extent. Therefore, in second example embodiment, the information on the anterior surface of the body, the shape of which is easy to recognize even in the dressed state in a further backward-bending posture, may be used to estimate the tilt of the pelvis and the shape of the trunk portion, and such estimation results may be used as the feature.

The feature calculating unit 103 estimates tilt of the pelvis in the following manner. That is, the feature calculating unit 103 determines the inclination of the tangent line to the edge of the anterior surface of the body at the position of the anterior superior iliac spine key point P5 as the tilt of the pelvis, and uses this inclination as a feature related to the pelvis. This slope refers to the inclination of the tangent line to the edge of the user's anterior surface of the body passing through the anterior superior iliac spine key point P5 in FIG. 5. This inclination is expressed, for example, as the angle θ3 indicating the inclination relative to the line segment connecting the prominent vertebra key point P3 and the hip joint key point P4, or the angle θ4 indicating the inclination relative to the line segment connecting the hip joint key point P4 and the knee joint key point P6.

In other words, in this example, the body information used to calculate the feature includes information indicating the edge of the user's anterior surface side (anterior surface of the body) edge in the backward-bending posture, and the feature calculating unit 103 calculates the feature at least relating to the pelvis based on the information indicating the edge of the anterior surface of the body. Also, the edge of the anterior surface of the body here differs from the edge described in Japanese Unexamined Patent Application Publication No. 2000-207568, and refers specifically to the entirety or part of the boundary between body region and non-body region in the image, which is located in the anterior surface of the body. The boundaries between the body region and the non-body region in the image may be obtained, for example, by estimating the body region and the non-body region in the image by using a learned region extraction model, respectively, and determining the boundary between these regions. Alternatively, the boundary described above may be derived from the one located in anterior surface of the body in an edge-enhanced image, which is obtained, for example, by applying an edge-enhancement filter, such as a Laplacian filter to the image. Alternatively, the user may provide the boundary described above via the input unit 107.

Alternatively, the feature calculating unit 103, not illustrated in the drawing, determines the inclination of the tangent line to the edge of the anterior surface of the body at the height of the hip joint key point P4 as the tilt of the pelvis, and uses this inclination as a feature related to the pelvis. Instead of this inclination, the inclination of a straight line passing through the anterior superior iliac spine key point P5 and the posterior superior iliac spine key point, not shown, can be used, although it is not the information about the anterior surface of the body.

In this manner, the body information used to calculate the feature can include information indicating the positions of the user's anterior superior iliac spine and posterior superior iliac spine in the backward-bending posture (referred to as “second position information” for convenience). The feature calculating unit 103 then calculates features at least related to the pelvis based on this second position information.

The feature calculating unit 103 can also calculate features using the shape of the trunk portion. Shape information on the anterior surface of the body is easier to acquire than shape information on the back surface of the body, but is easily affected by the shapes of the chest and abdomen. The shapes of the chest and abdomen that affect the anterior surface of the body can indicate, for example, how much degree the user is obese. Therefore, the feature calculating unit 103 may estimate the shape of the trunk portion by the following method and use the estimation result as the feature.

First, the feature calculating unit 103 acquires edges of the anterior and posterior surfaces of the trunk, respectively. Furthermore, the intersection points p_{(i, θ)}^{(f)} and p_{(i, θ)}^{(b)} of the straight line l_{(i, θ)} passing through the interior point pᵢ(i=1, . . . , n) of the line segment connecting the cervical vertebrae key point and hip joint key point, respectively, and the edges of the anterior and posterior surfaces of the trunk. Here, l_{(i, θ)} can be taken in an infinite number of ways, but for implementation purposes, the angle can be limited to 5-degree increments with θ as the angle from the horizontal line, for example, as follows.
74 ={0°, 5°, 10°, . . . , 175°}

The edges of the anterior and posterior surfaces of the trunk here differs from the edges described in Japanese Unexamined Patent Application Publication No. 2000-207568 as well as the edges of the anterior surface of the body, and refers specifically to the entirety or part of the boundary between body region and non-body region in the image, which is located in the anterior and posterior surface of the trunk, respectively. These can be obtained, for example, in the same way as the edge of the anterior surface of the body.

Next, the feature calculating unit 103 sets a pair of intersection points that minimize the distance between the two intersection points {(p_{(i, θ)}^{(f)}, p_{(i, θ)}^{(b)})}_{θ=0°, . . . , . . . , 175°)} to be the pair of representative points corresponding to pᵢ. Finally, the shape of the anterior surface of the body is determined by interpolating N (N is a positive integer) representative points {pᵢ^{(f)}}_{(i=1, . . . , n)} by fitting an nth order power function or spline function. The feature calculating unit 103 then uses this shape of the anterior surface of the body to calculate the curvature and the angle formed by the tangent lines as the feature.

In this manner, the feature calculating unit 103 can calculate the features related at least to the spine based on the information indicating the edge of the anterior surface of the body. The feature calculating unit 103 can also calculate features related at least to the spine based on the second position information described above.

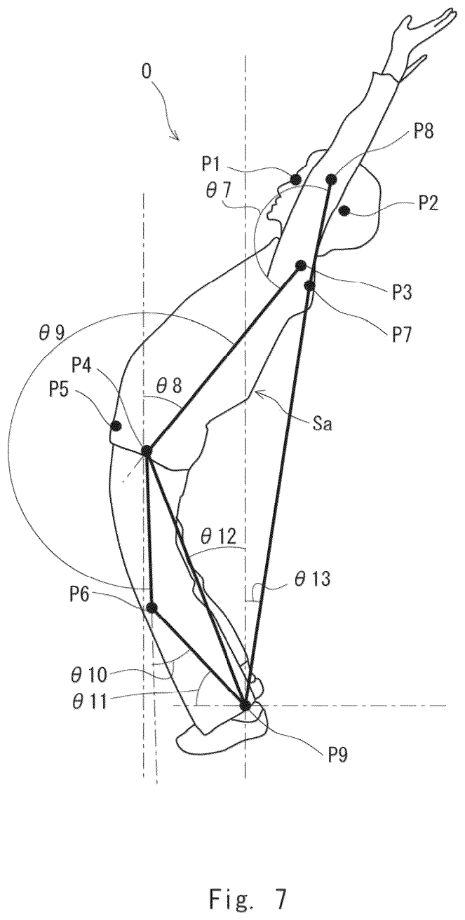

Referring now to FIG. 7, an example of the feature calculated by the feature calculating unit 103 using key point will be described. The feature calculating unit 103 can calculate the angle formed by the straight line passing through the prominent vertebra key point P3 and the hip joint key point P4, and the straight line passing through the shoulder joint key point P7 and the elbow joint key point P8, as shown by angle θ7, as a feature indicating the upper limb elevation angle. The feature calculating unit 103 can calculate the angle formed by the line segment connecting the prominent vertebra key point P3 and the hip joint key point P4 and the vertical line passing through the hip joint key point P4 in the image, as shown by angle θ8, as a feature indicating the backward-inclination angle of the trunk. The backward-inclination angle of the trunk represents the angle of the trunk relative to the vertical line.

The feature calculating unit 103 can also calculate the angle formed by the line segment connecting the prominent vertebra key point P3 and the hip joint key point P4, and the line segment connecting the hip joint key point P4 and the knee joint key point P6 as shown by the angle θ9, as a feature indicating the hip joint angle. This hip joint angle represents the angle of the hip joints relative to the trunk. The feature calculating unit 103 can also be calculated as a feature indicating the knee joints angle, as indicated by the angle θ10. Here, the angle θ10 represents the angle formed by the line segment connecting the knee joint key point P6 and the ankle joint key point P9 and the line segment connecting the hip joint key point P4 and the knee joint key point P6. The knee joint key point P6 on the image is a key point indicating the position of any of the following: the lower end of the femur, the patella, the upper end of the tibia, the upper end of the fibula, and the knee joints cleft.

The feature calculating unit 103 can calculate the angle formed by the line segment connecting the knee joint key point P6 and the ankle joint key point P9 and the horizontal line passing through the ankle joint key point P9 in the image, as shown by angle θ11, as a feature indicating the ankle joint angle. This ankle joint angle represents the angle of the ankle joints relative to the horizontal line. Alternatively, the feature calculating unit 103 may calculate the angle formed by the line segment connecting the knee joint key point P6 and the ankle joint key point P9, and the line segment connecting the key point indicating the position of the toe and the ankle joint key point P9, not shown in the drawing, as a feature indicating the ankle joint angle. The key point indicating the tiptoe can be, but is not limited to, the key point indicating the position of the distal phalanx of the third toe, for example.

The feature calculating unit 103 can calculate the angle formed by the line segment connecting the hip joint key point P4 and the ankle joint key point P9 and the vertical line passing through the ankle joint key point P9 in the image, as shown by angle θ12, as a feature indicating the position of the hip joints relative to the ankle joint. The feature calculating unit 103 can calculate the angle formed by the line segment connecting the shoulder joint key point P7 and the ankle joint key point P9 and the vertical line passing through the ankle joint key point P9 in the image, as shown by angle θ13, as a feature indicating the position of the shoulder joint relative to the ankle joint.

Next, the state-estimating unit 104 will be described.

The state-estimating unit 104 estimates the state of at least the spine in the backward-bending posture based on the feature calculated by the feature calculating unit 103.

The state-estimating unit 104 estimates the state of the spine in the backward-bending posture based on at least a feature including the feature of the inclination of the head relative to the trunk, such as at least one of the angles θ5 and θ6 as described above. In other words, the state of the spine is estimated based not on the shape of the spine itself, but on compensatory movements in parts other than the spine that occur in response to the mobility of the spine. The state-estimating unit 104 detects the compensation of the cervical vertebrae bending and extending according to the mobility of the upper thoracic spine by using the position and inclination of the head, thus indirectly estimating the state of the upper thoracic vertebrae. Specifically, at least one of the angles θ5 and θ6 is obtained to estimate the state of the upper thoracic spine based, for example, on the reference value of the head angle or the neck angle.

The state-estimating unit 104 can also improve the accuracy of estimating the state of the upper thoracic spine by using the feature other than the head angle and the neck angle, or estimate the state of parts of the spine other than the upper thoracic spine, such as the lower thoracic spine, lumbar spine, and lumbosacral transition region. The target of estimation may include the hip joints, the upper extremity, the scapular spine, the anterior superior iliac spine, the knee joints, and the ankle joints.

In addition, the state-estimating unit 104 has the reference value list 111 that maps each feature to its reference value stored in the storage unit 110. The reference value list 111 is an example of a correspondence table, described in first example embodiment, that maps feature to the state of the spine and the like in the backward-bending posture. Here, the reference value is the range of values that the feature can take when the body parts, such as the spine and the like, is normal. Then, the state-estimating unit 104 refers to the reference value list 111 based on the feature input from the feature calculating unit 103 and estimates the state of the spine and the like according to whether the feature satisfies the reference values or not. For example, if a certain feature satisfies the reference value, the state-estimating unit 104 estimates the state related to the feature is moderate. Likewise, if it exceeds the reference value, it is estimated that the backward-bending extension, for example, is excessive, and if it falls below the reference value, it is estimated that the backward-bending extension, for example, is limited.

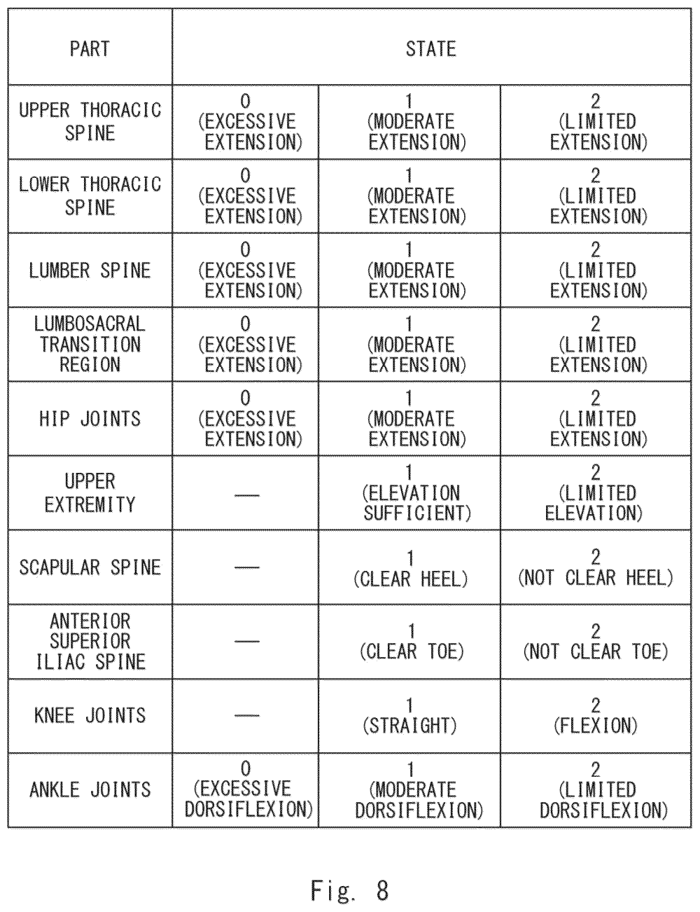

Referring now to FIG. 8, examples of the estimation result on each part that is output by the state-estimating unit 104 will be shown. Here is an example where state labels are output as the estimation results. For example, as shown in FIG. 8, the state-estimating unit 104 estimates that the upper thoracic spine is in moderate extension if the feature indicating the upper thoracic spine feature is within the reference values and outputs the state label “1”. Examples of the feature of the upper thoracic spine include, for example, the backward-inclination angle of the trunk. If the feature of the upper thoracic spine exceeds the reference value, the state-estimating unit 104 estimates that it is an excessive extension and outputs the state label “0”. If the feature of the upper thoracic spine is below the reference value, the state-estimating unit 104 estimates that extension is limited and outputs the state label “2”.

The lower thoracic spine, the lumbar spine, the lumbosacral transition region, and the hip joints are processed in the same manner as the upper thoracic spine. Examples of the features indicating the features of the lower thoracic spine and the lumbosacral transition region both include, for example, the backward-inclination angle of the trunk. Examples of the feature of the lumbar spine include, for example, the backward-inclination angle of the trunk. Examples of the feature of the hip joints include, for example, the angle of the hip joints.

If the feature of the ankle joints is within the reference values, the state-estimating unit 104 estimates that the ankle joints are moderately dorsiflexed and outputs the state label “1”. Examples of the feature of the ankle joints include, for example, the angle of the ankle joints. If the feature of the ankle joints is below the reference value, the state-estimating unit 104 estimates that dorsiflexion is excessive and outputs the state label “0”, while if the feature is above the reference value, the state-estimating unit 104 estimates that dorsiflexion is limited and outputs the state label “2”.

For the upper extremity, the scapular spine, the anterior superior iliac spine, and the knee joints, each reference value can be kept as one threshold. If the feature of the upper extremity is equal to or higher than the reference value, the state-estimating unit 104 estimates that elevation is sufficient and outputs the state label “1”, while if it is below the reference value, the state-estimating unit 104 estimates that elevation is limited and outputs the state label “2”. Examples of the feature of the upper extremity include, for example, the upper limb elevation angle. If the feature of the scapular spine is greater than or equal to the reference value, the state-estimating unit 104 estimates that the horizontal position exceeds the horizontal position of the heel and outputs the state label “1”, while if it is less than the reference value, then estimates that the horizontal position does not exceed the horizontal position of the heel and outputs the state label “2”. Examples of the feature of the scapular spine include, for example, the position of the shoulder joints relative to the ankle joints.

If the feature of the anterior superior iliac spine is equal to or higher than the reference value, the state-estimating unit 104 estimates that the horizontal position exceeds the horizontal position of the toe and outputs the state label “1”, while if the feature is less than the reference value, the state-estimating unit 104 estimates that the horizontal position does not exceed the horizontal position of the toe and outputs the state label “2”. Examples of the feature of the anterior superior iliac spine include, for example, the position of the hip joints position relative to the ankle joints. If the feature of the knee joints is equal to or smaller than the reference value, the state-estimating unit 104 estimates that there is no flexion and outputs the state label “1”, while if the feature exceeds the reference value, it estimates that there is flexion and outputs the state label “2”. The feature of the knee joints include, for example, the knee joints angle.

The state-estimating unit 104 may also use a state estimation model as a learning model generated by machine learning, input the feature to the state estimation model, and obtain estimation results of the state as its output. When the state-estimating unit 104 performs estimation using a state estimation model, this state estimation model can be stored in advance in the storage unit 110. The state estimation model can be, for example, a machine-learned model using a dataset that maps features to state labels such as “excessive,” “moderate,” or “limited” as correct data.

The state-estimating unit 104 inputs the estimation results thus obtained to the image-generating unit 105.

The image-generating unit 105 generates an estimation result display image to be displayed by the display unit 108 based on the estimation results input from the state-estimating unit 104. The estimation result image can include a normalized image of the image captured by the image-capturing unit 101. The image-generating unit 105 then inputs the generated image to the display unit 108.

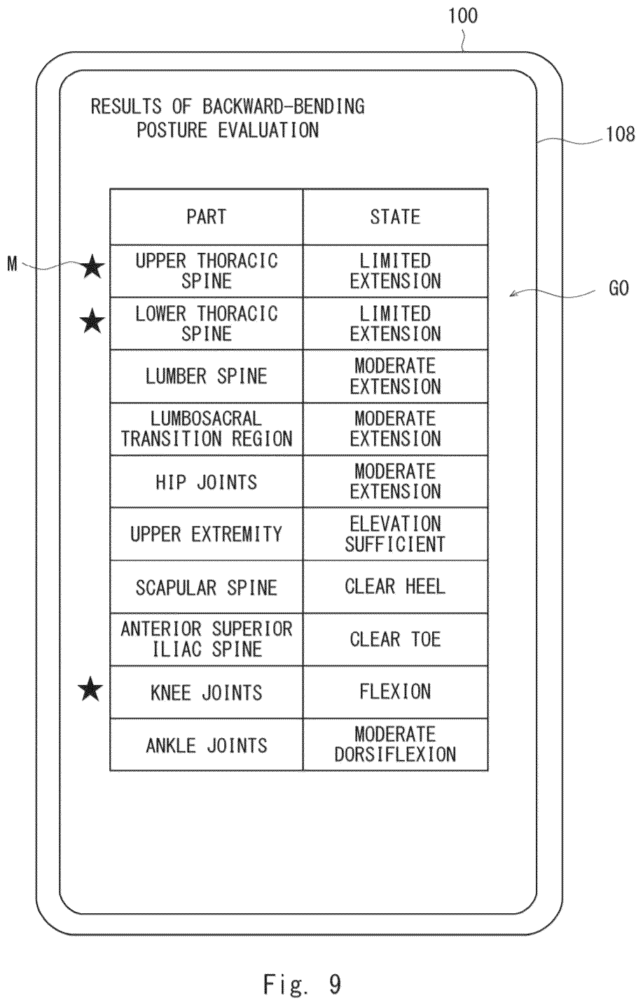

The display unit 108 displays the estimation result display image input from the image-generating unit 105. In other words, the display unit 108 can display at least part of the results estimated by the state-estimating unit 104. The display unit 108 is composed of various display means such as LCD (Liquid Crystal Display), LED (Light Emitting Diode), and so on. FIGS. 9 through 12 show various examples of the estimation result display images shown on the display unit 108.

In the example shown in FIG. 9, the display unit 108 of the posture evaluation apparatus 100 displays an image portion G0 showing the estimation results of the backward-bending posture estimation, or the evaluation results of the backward-bending posture. The image portion G0 contains a list of part names and the corresponding evaluation results of the state. In the image portion G0, the estimation results indicating “not normal” are emphasized with a star mark M. It is recommended to display the results be displayed in display forms different from that for normal parts, for example, by being emphasized in bold or red letters instead.

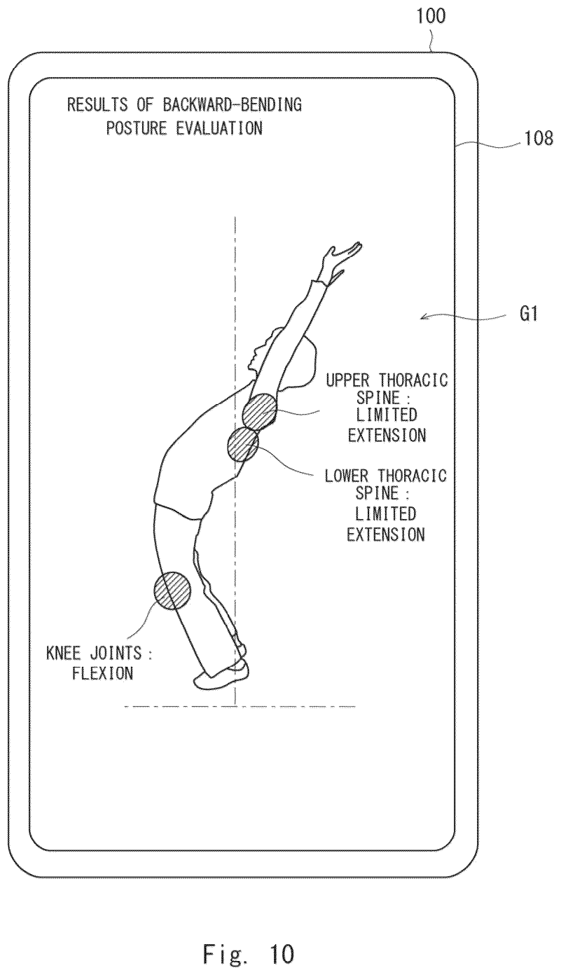

In the example illustrated in FIG. 10, the display unit 108 of the posture evaluation apparatus 100 displays an image portion G1 in which the estimation results are superimposed on the image captured by the image-capturing unit 101. In image portion G1, the rough area of the part where the estimation results indicate “not normal” is emphasized with a state of being surrounded by a graphic such as an oval or circle. The image portion G1 also includes a statement explaining that each area is not normal, such as “upper thoracic spine: limited extension,” “lower thoracic spine: limited extension,” or “knee joints: flexion” for each region.

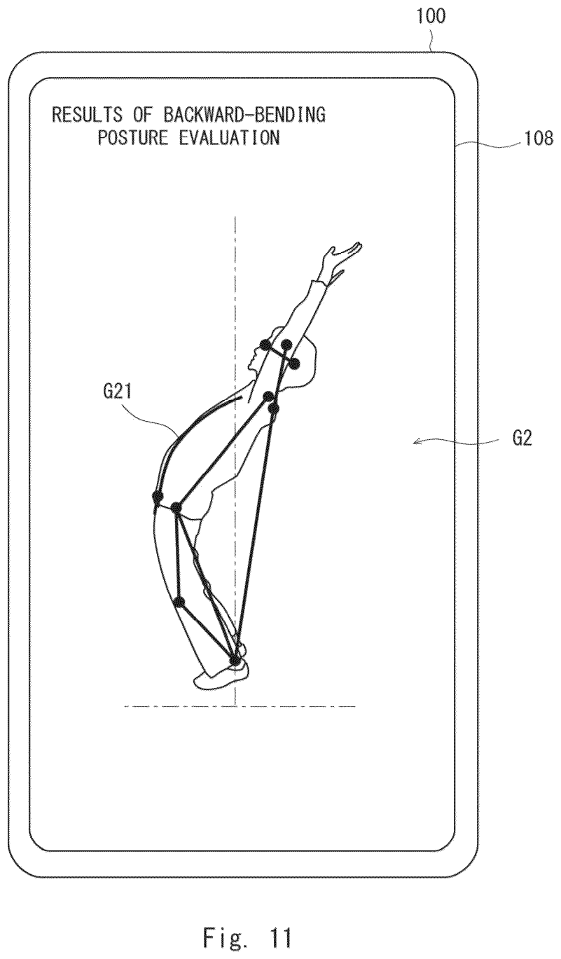

In the example shown in FIG. 11, the display unit 108 of the posture evaluation apparatus 100 displays an image portion G2 in which key points and line segments connecting the key points related to the calculation of angles, etc., are superimposed on the image captured by the image-capturing unit 101. In the image portion G2, the shape G21 of the anterior surface of the trunk estimated by the feature calculating unit 103 may also be shown. In addition, at least one of the image portions G0 in FIG. 9 and image portion G1 in FIG. 10 may also be displayed on the display unit 108 together with the image portion G2.

The display unit 108 may also display the image for correction input from the image-generating unit 105, such as displaying the image portion G2 in a manner that accepts correction. This allows, for example, if the key points extracted by the body keypoint extracting unit 102 are wrong, the user can correct them by dragging the target key points displayed on the screen for correction.

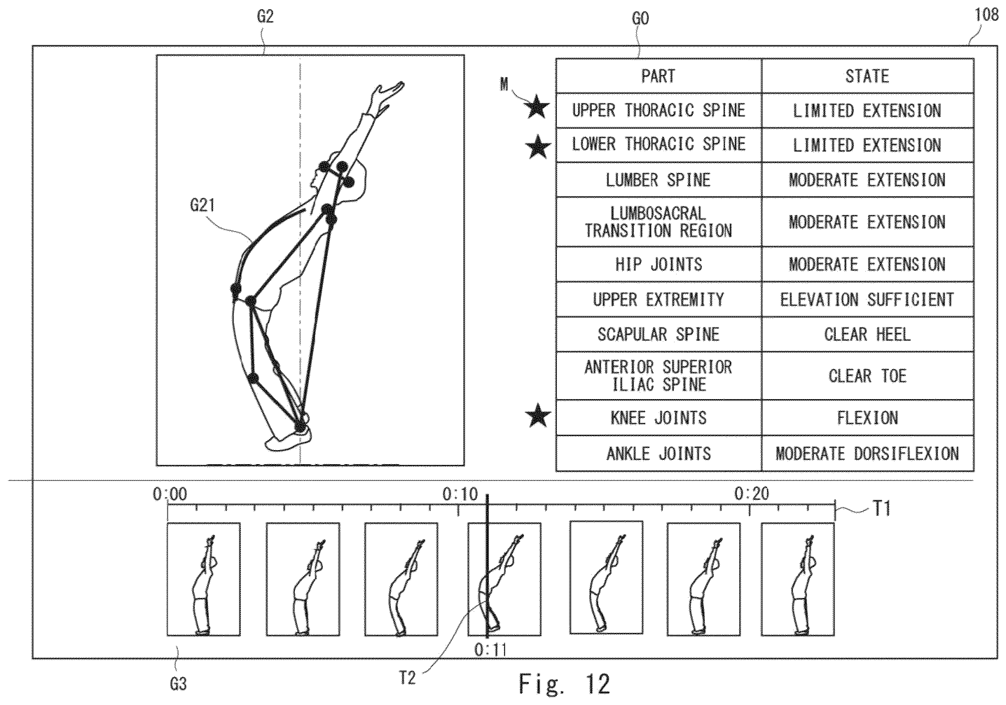

In the example shown in FIG. 12, the image selection area G3 is displayed on the display unit 108 of the posture evaluation apparatus 100. The image selection area G3 is an image area in which multiple thumbnail images of the video input from the image-capturing unit 101 are arranged along the time scale T1, and a specifying bar T2 movably displayed along the time scale T1 for specifying the time point when the posture evaluation is to be performed. The image-generating unit 105 should generate the image selection area G3 to be displayed by the display unit 108 based on the image input from the image-capturing unit 101.

The image-generating unit 105 generates the image portion G2 by superimposing the estimation results on the image at the time point specified by the user in the image selection area G3 displayed on the display unit 108. The image-generating unit 105 generates an image portion G0 by superimposing the estimation results on the image at the time point specified by the user in the image selection area G3 displayed on the display unit 108. The image-generating unit 105 then inputs the generated image selection area G3, image portion G2, and image portion G0 to the display unit 108.

The display unit 108 displays the image selection area G3, image portion G2, and image portion G0 input from the image-generating unit 105. In the example illustrated in FIG. 12, the image selection area G3 is displayed on the lower side of the display unit 108 of the posture evaluation apparatus 100, the image portion G2 is displayed on the upper left side of the display unit 108, and the image portion G0 is displayed on the upper right side of the display unit 108. In the example illustrated in FIG. 12, in the image selection area G3, the user moves the specifying bar T2, and 0 minutes and 11 seconds are specified as the time point when the posture evaluation is to be performed.

The posture evaluation apparatus 100 may be equipped with a modifying unit that modifies at least one of the display form and the display content of the results based on the subject person to be presented, to whom the estimation results are to be presented. The modifying unit may execute at least one of the display form and the display contents upon acceptance of a user operation to specify a subject person to be presented from the input unit 107 according to the user operation, or may execute the same according to user information of a user using the posture evaluation apparatus 100. For example, if the subject person to be presented is a user, the display form illustrated in FIG. 10, which is easier to understand, can be applied, and if it is a trainer or the like, the display form shown in FIG. 9, FIG. 11, or FIG. 12, which shows more information, can be applied. Of course, the contents and display form of display on the display unit 108 are not limited to those illustrated in FIGS. 9 through 12.

The communicating unit 106 communicates with external servers, other terminal apparatus and the like. The communicating unit 106 may be equipped with an antenna (not illustrated) for wireless communication or an interface such as a Network Interface Card (NIC) or the like for wired communication.

The input unit 107 accepts operation instructions from the user. The input unit 107 may be configured by a keyboard or by a touch panel display device. The input unit 107 may be configured by a keyboard or a touch panel connected to a main body of the posture evaluation apparatus 100.

The storage unit 110 stores the reference value list 111, the body keypoint database 112, the body keypoint extraction model 113, and others. The storage unit 110 can include a nonvolatile memory (e.g., ROM (Read Only Memory)) in which various programs and various data necessary for processing are fixedly stored. The storage unit 110 may be the type using a Hard Disk Drive (HDD) or Solid-State Drive (SSD). In addition, the storage unit 110 can include a volatile memory, such as RAM (Random Access Memory), which is used as a work area. The program described above may be read from a portable recording medium such as an optical disc, a semiconductor memory or the like, or may be downloaded from a server apparatus on the network.

The reference value list 111 is a list that maps each feature to its reference value in the backward-bending posture. The reference value list 111 can be prepared for each posture other than the backward-bending posture, such as forward-bending posture and standing posture.

The body keypoint database 112 is a database which maps the images to the position information of the key points as correct labels for a plurality of images obtained by capturing the image of the side surface of the body in the backward-bending posture.

The body keypoint extraction model 113 is a learning model that extracts posture position information of the key points from images obtained by capturing the image of the side surface of the body in the backward-bending posture. In other words, the body keypoint extraction model 113 is a learning model that uses as input an image obtained by capturing the side surface of the user's body in a backward-bending posture and outputs inferred position information of the key points. In this specification, machine learning may be deep learning, but is not specifically limited thereto.

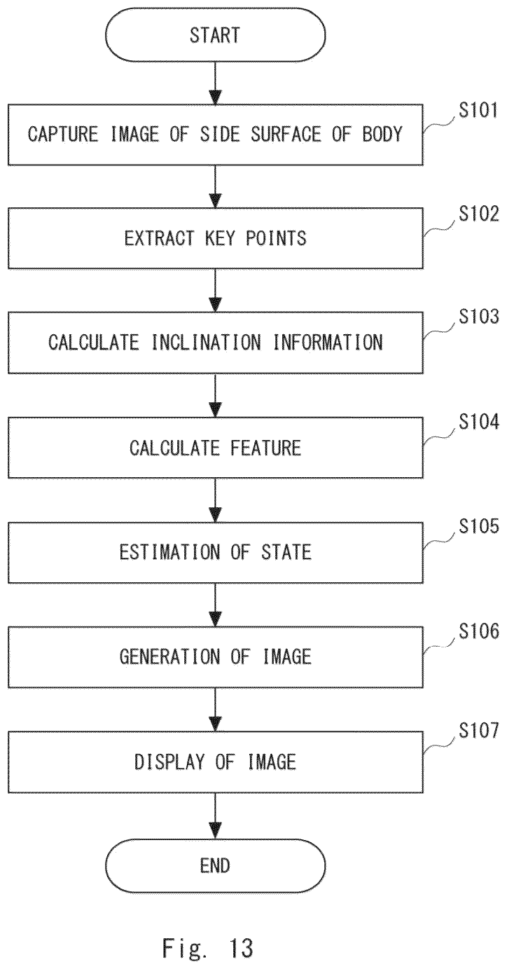

Referring to FIG. 13, the posture evaluation method performed by the posture evaluation apparatus 100 is described. Although only a brief flow is described here, the various examples described above can be applied.

First, the image-capturing unit 101 captures images of the side surface of the body (Step S101), and inputs the captured images to the body keypoint extracting unit 102. Next, the body keypoint extracting unit 102 extracts the position information of the key points from the image captured by the image-capturing unit 101 in Step S101 (Step S102). Next, the body keypoint extracting unit 102 calculates information indicating at least the inclination of the trunk and the inclination of the head based on the extracted position information and inputs the information to the feature calculating unit 103 (Step S103).

Next, the feature calculating unit 103 calculates feature including at least the feature of the inclination of the head in relation to the trunk based on information input from the body keypoint extracting unit 102 (Step S104). The feature calculating unit 103 then inputs the calculated feature to the state-estimating unit 104.

Next, the state-estimating unit 104 estimates the state of at least the spine, for example, the upper thoracic spine, the lower thoracic spine, and the lumbar spine based on the feature input from the feature calculating unit 103 (Step S105). The state-estimating unit 104 then inputs the estimation results to the image-generating unit 105.

Next, the image-generating unit 105 generates an image to be displayed on the display unit 108 based on the images captured in Step S101 and the state of the upper thoracic spine, the lower thoracic spine, the lumbar spine, or the like estimated in Step S105 (Step S106). The image is then input by the image- generating unit 105 to the display unit 108. Next, the display unit 108 displays the image generated in Step S106 (Step S107) to end this process. Instead of processing Step S102, each key point may be specified by the user operating the input unit 107 via an image captured by the image-capturing unit 101 and displayed on the display unit 108.

As with first example embodiment, second example embodiment produces an effect that backward-bending posture can be evaluated relatively easily and with high degree of accuracy even when the user is wearing clothes, and in addition, by performing image-based evaluation, expensive specialized equipment does not need to be used.

In addition, according to second example embodiment, as key points that are relatively easy to extract such as eyes and ears are used, improvement of the accuracy of evaluation is achieved. According to second example embodiment, with information indicating the position of the head relative to the trunk included as body information, reduction of the processing load for calculating feature and precise calculation of the feature are achieved. According to second example embodiment, with information indicating the edge of the front surface of the user's body included in the body information, information that is relatively easy to recognize from the image can be used, so that improvement of the accuracy of the evaluation and the evaluation of the pelvis with high degree of accuracy are achieved. According to second example embodiment, with information indicating the position of the anterior superior iliac spine and posterior superior iliac spine included in the body information, evaluation with high degree of accuracy is achieved for not only the spine but also the pelvis.

In addition, the posture evaluation apparatus 100 is equipped with a body keypoint extracting unit 102, which extracts key points from images captured by the image-capturing unit 101, so that the need for the user to specify key points on said images is eliminated.

The body keypoint extracting unit 102 may also use the learned body keypoint extraction model 113 to extract key points represented by three-dimensional coordinates defined in the x-direction being the left and right or horizontal direction, the y-direction being the up and down or vertical direction, and the z-direction being the depth direction of the two-dimensional image captured by the image-capturing unit 101. This allows for more precise posture evaluation.

The user can visually recognize the state of the posture by displaying the estimation result display image that displays the image captured by the image-capturing unit 101 and the estimation results by the state-estimating unit 104 displayed on the display unit 108. The posture evaluation apparatus 100 may be configured to change the display form and the like in the display unit 108 according to the subject person to be presented, so that the display can be tailored to the level of understanding of the subject person to be presented.

In second example embodiment, though the processing only from images captured from one side surface has been described, images captured from the other side surface can also be used for at least one of extraction of key points, calculation of features, and estimation of the spine and the like. In the simplest example, the intended results can be obtained, for example, by calculating the average of both the left and right sides in any of those processes. In addition, by simply displaying images from other angles, such as the front and rear views when the user is presented with the images, the user can confirm information about posture that cannot be obtained by simply checking the sides of the body, such as whether the left and right sides of the body are moving equally. However, images from the other directions described above may also be used for at least one of extraction of key points, calculation of features, and estimation of the state of the spine and the like.

### Third Example Embodiment

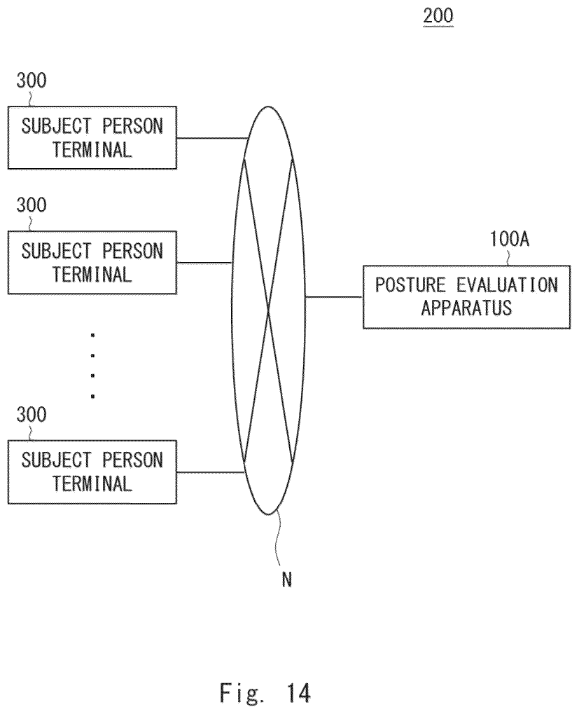

Referring next to FIG. 14, a configuration example of a posture evaluation system 200 will be described. The posture evaluation system 200 includes a posture evaluation apparatus 100A and a subject person terminal 300 that can communicate with the posture evaluation apparatus 100A, as illustrated in FIG. 14. The posture evaluation apparatus 100A and the subject person terminal 300 can communicate via the network N. As illustrated in FIG. 14, one or more subject person terminals 300 may be configured to be able to communicate with the posture evaluation apparatus 100A. Examples of the subject person terminal 300 include a smartphone, a tablet terminal, a personal computer owned by the subject person.

The posture evaluation apparatus 100A acquires an image of the side of the body of the subject person in the backward-bending posture from the subject person terminal 300. Therefore, the posture evaluation apparatus 100A differs from the posture evaluation apparatus 100 in FIG. 2 in that the image-capturing unit 101 may be omitted. The estimation result display image created by the image-generating unit 105 of the posture evaluation apparatus 100A may be transmitted to the subject person terminal 300 and displayed on the display unit (not illustrated) of the subject person terminal 300.

The subject person terminal 300 is equipped with an image-capturing unit (not illustrated) that captures images of the side of the body of the subject person in the backward-bending posture. The subject person terminal 300 transmits the captured images to the posture evaluation apparatus 100A.

### Other Example Embodiments

In the example embodiments described thus far, the present disclosure is described as, but not limited thereto, a hardware configuration. The present disclosure can also be realized by making a CPU (Central Processing Unit) execute a computer program for the processing procedures described in the flowchart in FIG. 13 and other example embodiments.

The program includes instructions (or software codes) that, when loaded into a computer, cause the computer to perform one or more of the functions described in the embodiments. The program may be stored in a non-transitory computer readable medium or a tangible storage medium. By way of example, and not limitation, non-transitory computer readable media or tangible storage media can include a random-access memory (RAM), a read-only memory (ROM), a flash memory, a solid-state drive (SSD) or other memory technologies, CD-ROM, digital versatile disc (DVD), Blu-ray disc or other optical disc storage, magnetic cassettes, magnetic tape, magnetic disk storage or other magnetic storage devices. The program may be transmitted on a transitory computer readable medium or a communication medium. By way of example, and not limitation, transitory computer readable media or communication media can include electrical, optical, acoustical, or other form of propagated signals.

The present disclosure is not limited to the example embodiments described above, and may be modified as appropriate without departing from the gist of the present disclosure.

While the disclosure has been particularly shown and described with reference to example embodiments thereof, the disclosure is not limited to these example embodiments. It will be understood by those of ordinary skill in the art that various changes in form and details may be made therein without departing from the spirit and scope of the present disclosure as defined by the claims. Each example embodiment can be combined with other example embodiment as desirable by one of ordinary skill in the art.

Each drawing is merely an example to illustrate one or more example embodiments. Each drawing may not be associated with only one particular example embodiment, but may be associated with one or more other example embodiments. As one skilled in the art will understand, the various features or steps described with reference to any one of the drawings can be combined with features or steps illustrated in one or more other drawings, for example, to produce an example embodiment not explicitly illustrated or described. Not all of the features or steps illustrated in any one of the drawings to describe the example embodiment are necessarily required, and some features or steps may be omitted. The order of the steps described in any of the drawings may be modified as appropriate.

The whole or part of the example embodiments disclosed above can be described as, but not limited to, the following supplementary notes.

### Supplementary Note 1

A posture evaluation apparatus including:
a feature calculating unit configured to calculate a feature including at least a feature of an inclination of a head relative to a trunk, based on body information including information indicating at least an inclination of the trunk and an inclination of the head in a body of a subject person in a backward-bending posture in which the subject person is bent backward; and
a state-estimating unit configured to estimate a state of at least a spine in the backward-bending posture, based on the feature.

### Supplementary Note 2

The posture evaluation apparatus according to Supplementary Note 1, wherein the body information includes an image of a side surface of the body of the subject person being captured in the backward-bending posture or information extracted from the image.

### Supplementary Note 3

The posture evaluation apparatus according to Supplementary Note 1 or 2, wherein the body information includes information indicating a position of the head relative to the trunk in the body of the subject person in the backward-bending posture, and
the feature includes a feature of the position of the head relative to the trunk.

### Supplementary Note 4

The posture evaluation apparatus according to Supplementary Note 1 or 2, wherein
the body information includes first position information indicating positions of at least a cervical vertebrae, hip joints, eyes, and ears of the subject person in the backward-bending posture, and
the feature calculating unit calculates an inclination of the trunk and an inclination of the head, based on the first position information, and calculates the feature including at least the feature of the inclination of the head relative to the trunk by using the calculated inclination of the trunk and the calculated inclination of the head.

### Supplementary Note 5

The posture evaluation apparatus according to Supplementary Note 3, wherein
the body information includes first position information indicating positions of at least a cervical vertebrae, hip joints, eyes, and ears of the subject person in the backward-bending posture, and
the feature calculating unit calculates an inclination of the trunk, an inclination of the head, and a position of the head relative to the trunk, based on the first position information, and calculates a feature indicating at least the inclination and a position of the head relative to the trunk by using the calculated inclination of the trunk, the calculated inclination of the head, and the calculated position of the head relative to the trunk.

### Supplementary Note 6

The posture evaluation apparatus according to any one of Supplementary Notes 1 to 5, wherein
the body information includes information indicating an edge on an anterior surface side of the subject person in the backward-bending posture, and
the feature calculating unit calculates at least a feature relating to a pelvis or a spine, based on the information indicating the edge on the anterior surface side.

### Supplementary Note 7

The posture evaluation apparatus according to any one of Supplementary Notes 1 to 6, wherein the body information includes second position information indicating positions of an anterior superior iliac spine and an posterior superior iliac spine of the subject person in the backward-bending posture, and
the feature calculating unit calculates at least a feature relating to a pelvis or a spine based on the second position information.

### Supplementary Note 8

The posture evaluation apparatus according to any one of Supplementary Notes 1 to 7, further including:

a display unit configured to display at least a part of a result estimated by the state-estimating unit; and a modifying unit configured to modify at least one of a display form and a display content of the result, based on a subject person to be presented to whom the result is to be presented.

### Supplementary Note 9

A posture evaluation system including:
a posture evaluation apparatus; and
a terminal apparatus configured to be able to communicate with the posture evaluation apparatus, wherein the posture evaluation apparatus includes:
a feature calculating unit configured to calculate a feature including at least a feature of an inclination of a head relative to a trunk, based on body information including information indicating at least an inclination of the trunk and an inclination of the head in a body of a subject person in a backward-bending posture in which the subject person is bent backward, the body information being acquired by the terminal apparatus and
a state-estimating unit configured to estimate a state of at least the spine in the backward-bending posture based on the feature.

### Supplementary Note 10

A posture evaluation method including, by a posture evaluation apparatus:
calculating a feature including at least a feature of an inclination of a head relative to a trunk, based on body information including information indicating at least an inclination of the trunk and an inclination of the head in a body of a subject person in a backward-bending posture in which the subject person is bent backward, and
estimating a state of at least a spine in the backward-bending posture, based on the feature.

### Supplementary Note 11

A program causing a computer to execute posture evaluation processing of:
calculating a feature including at least a feature of an inclination of a head relative to a trunk based on body information including information indicating at least an inclination of the trunk and an inclination of the head in a body of a subject person in a backward-bending posture in which the subject person is bent backward, and
estimating a state of at least a spine in the backward-bending posture, based on the feature.

Some or all of elements (e.g., structures and functions) specified in Supplementary Notes 2 to 8 dependent on Supplementary Note 1 may also be dependent on Supplementary Note 9, Supplementary Note 10, and Supplementary Note 11 in dependency similar to that of Supplementary Notes 2 to 8 on Supplementary Note 1. Some or all of elements specified in any of Supplementary Notes may be applied to various types of hardware, software, and recording means for recording software, systems, and methods.

According to the present disclosure, a posture evaluation apparatus, a posture evaluation system, a posture evaluation method, and a program capable of suitably evaluating backward-bending postures may be provided

What is claimed is:

1. A posture evaluation apparatus comprising:
at least one memory storing instructions; and
at least one processor configured to execute the instructions to do posture evaluation process, wherein the posture evaluation process includes:
calculating a feature including at least a feature of an inclination of a head relative to a trunk, based on body information including information indicating at least an inclination of the trunk and an inclination of the head in a body of a subject person in a backward-bending posture in which the subject person is bent backward; and
estimating a state of at least a spine in the backward-bending posture, based on the feature.

2. The posture evaluation apparatus according to claim 1, wherein the body information includes an image of a side surface of the body of the subject person being captured in the backward-bending posture or information extracted from the image.

3. The posture evaluation apparatus according to claim 1, wherein the body information includes information indicating a position of the head relative to the trunk in the body of the subject person in the backward-bending posture, and
the feature includes a feature of the position of the head relative to the trunk.

4. The posture evaluation apparatus according to claim 1, wherein
the body information includes first position information indicating positions of at least a cervical vertebrae, hip joints, eyes, and ears of the subject person in the backward-bending posture, and
the calculating includes calculating an inclination of the trunk and an inclination of the head, based on the first position information, and calculating the feature including at least the feature of the inclination of the head relative to the trunk by using the calculated inclination of the trunk and the calculated inclination of the head.

5. The posture evaluation apparatus according to claim 3, wherein
the body information includes first position information indicating positions of at least a cervical vertebrae, hip joints, eyes, and ears of the subject person in the backward-bending posture, and
the calculating includes calculating an inclination of the trunk, an inclination of the head, and a position of the head relative to the trunk, based on the first position information, and calculating a feature indicating at least the inclination and a position of the head relative to the trunk by using the calculated inclination of the trunk, the calculated inclination of the head, and the calculated position of the head relative to the trunk.

6. The posture evaluation apparatus according to claim 1, wherein
the body information includes information indicating an edge on an anterior surface side of the subject person in the backward-bending posture, and
the calculating includes calculating at least a feature relating to a pelvis or a spine, based on the information indicating the edge on the anterior surface side.

7. The posture evaluation apparatus according to claim 1, wherein the body information includes second position information indicating positions of an anterior superior iliac spine and an posterior superior iliac spine of the subject person in the backward-bending posture, and
the calculating includes calculating at least a feature relating to a pelvis or a spine based on the second position information.

8. The posture evaluation apparatus according to claim 1, wherein the posture evaluation process further includes:
displaying at least a part of a result estimated by the estimating on a display; and
modifying at least one of a display form and a display content of the result, based on a subject person to be presented to whom the result is to be presented.

9. A posture evaluation method including, by a posture evaluation apparatus:
calculating a feature including at least a feature of an inclination of a head relative to a trunk, based on body information including information indicating at least an inclination of the trunk and an inclination of the head in a body of a subject person in a backward-bending posture in which the subject person is bent backward, and
estimating a state of at least a spine in the backward-bending posture, based on the feature.

10. The posture evaluation method according to claim 9, wherein the body information includes an image of a side surface of the body of the subject person being captured in the backward-bending posture or information extracted from the image.

11. The posture evaluation method according to claim 9, wherein the body information includes information indicating a position of the head relative to the trunk in the body of the subject person in the backward-bending posture, and
the feature includes a feature of the position of the head relative to the trunk.

12. The posture evaluation method according to claim 9, wherein
the body information includes first position information indicating positions of at least a cervical vertebrae, hip joints, eyes, and ears of the subject person in the backward-bending posture, and
the calculating includes calculating an inclination of the trunk and an inclination of the head, based on the first position information, and calculating the feature including at least the feature of the inclination of the head relative to the trunk by using the calculated inclination of the trunk and the calculated inclination of the head.

13. The posture evaluation method according to claim 11, wherein
the body information includes first position information indicating positions of at least a cervical vertebrae, hip joints, eyes, and ears of the subject person in the backward-bending posture, and
the calculating includes calculating an inclination of the trunk, an inclination of the head, and a position of the head relative to the trunk, based on the first position information, and calculating a feature indicating at least the inclination and a position of the head relative to the trunk by using the calculated inclination of the trunk, the calculated inclination of the head, and the calculated position of the head relative to the trunk.

14. The posture evaluation method according to claim 9, wherein
the body information includes information indicating an edge on an anterior surface side of the subject person in the backward-bending posture, and
the calculating includes calculating at least a feature relating to a pelvis or a spine, based on the information indicating the edge on the anterior surface side.

15. A non-transitory computer readable medium configured to store a program causing a computer to execute posture evaluation processing of:
calculating a feature including at least a feature of an inclination of a head relative to a trunk based on body information including information indicating at least an inclination of the trunk and an inclination of the head in a body of a subject person in a backward-bending posture in which the subject person is bent backward, and
estimating a state of at least a spine in the backward-bending posture, based on the feature.

16. The non-transitory computer readable medium according to claim 15, wherein the body information includes an image of a side surface of the body of the subject person being captured in the backward-bending posture or information extracted from the image.

17. The non-transitory computer readable medium according to claim 15, wherein the body information includes information indicating a position of the head relative to the trunk in the body of the subject person in the backward-bending posture, and
the feature includes a feature of the position of the head relative to the trunk.

18. The non-transitory computer readable medium according to claim 15, wherein
the body information includes first position information indicating positions of at least a cervical vertebrae, hip joints, eyes, and ears of the subject person in the backward-bending posture, and
the calculating includes calculating an inclination of the trunk and an inclination of the head, based on the first position information, and calculating the feature including at least the feature of the inclination of the head relative to the trunk by using the calculated inclination of the trunk and the calculated inclination of the head.

19. The non-transitory computer readable medium according to claim 17, wherein
the body information includes first position information indicating positions of at least a cervical vertebrae, hip joints, eyes, and ears of the subject person in the backward-bending posture, and
the calculating includes calculating an inclination of the trunk, an inclination of the head, and a position of the head relative to the trunk, based on the first position information, and calculating a feature indicating at least the inclination and a position of the head relative to the trunk by using the calculated inclination of the trunk, the calculated inclination of the head, and the calculated position of the head relative to the trunk.

20. The non-transitory computer readable medium according to claim 15, wherein
the body information includes information indicating an edge on an anterior surface side of the subject person in the backward-bending posture, and
the calculating includes calculating at least a feature relating to a pelvis or a spine, based on the information indicating the edge on the anterior surface side.
